(19) United States
(12) Patent Application Publication
(10) Pub. No.: US 2017/0253919 A1
(43) Pub. Date: Sep. 7, 2017

(54) PROBE SETS AND METHODS FOR ANALYZING HYBRIDIZATION

(71) Applicant: VITO NV, Mol (BE)

(72) Inventor: Jef Hooyberghs, Mol (BE)

(21) Appl. No.: 15/511,366

(22) PCT Filed: Sep. 17, 2015
(86) PCT No.: PCT/EP2015/071305

§ 371 Date: Mar. 15, 2017

(30) Foreign Application Priority Data
Sep. 17, 2014 (EP) 14185210.3

(51) Int. Cl. C12Q 1/68

(52) U.S. Cl. CPC C12Q 1/6832; C12Q 1/6837

(57) ABSTRACT

Provided herein are methods for analyzing hybridization between a target polynucleotide in a sample solution and probes bound to a surface, such as probes of a microarray. The methods involve contacting said sample solution to at least two probe sets, which are characterized in that the probes within each probe set have a hybridization sequence of a fixed length. Probes of different probe sets have hybridization sequences of different lengths. A comparison between the data obtained for the different probe sets allows for increasing the dynamic range of hybridization methods.

### FIELD OF THE INVENTION

The present invention relates to methods for detecting a target polynucleotide based on hybridization, and related kits and computer programs. The present methods allow for increasing the dynamic range of hybridization-based methods.

### BACKGROUND OF THE INVENTION

The accurate detection of DNA sequence variations such as single nucleotide variations (SNVs) and somatic mutations is of great interest for a variety of research domains. For example, single nucleotide polymorphisms (SNPs) are DNA sequence variations between members of the same species characterized by a single nucleotide difference and account for the majority of the genome variations between individuals of said species. SNPs are functionally highly relevant and their detection is of importance for a multitude of research and application domains, including genome-wide association studies, personalized molecular diagnostics and forensic identification.

Techniques for the detection of sequence variations such as SNPs can mainly be classified as either hybridization-based techniques or enzyme-based techniques such as PCR and sequencing. The use of hybridization-based techniques is appealing due to their inherent simplicity. These techniques typically involve providing a nucleic acid probe to hybridize to the perfectly matching DNA target with a high efficiency, while having a low efficiency for targets containing a single nucleotide variation. WO2011/035801 describes a method for analyzing hybridization, involving the analysis of hybridization intensities for different probes as a function of hybridization free energy.

However, finding the probe design and experimental conditions for obtaining an optimal specificity can present a challenge. The degree of hybridization of the target to a certain probe is typically measured via the intensity of a marker such as a fluorescence marker, and may therefore be referred to as “hybridization intensity”. The measured hybridization intensity in any system depends on the concentration of the target and on the probe-target affinity, the latter being determined by the hybridization free energy ΔG. On the lower end of the intensity spectrum, the signal intensity can drop below detection limit l₀ (insufficient probes hybridizing to a target) and on the high end the intensity may be saturated (virtually all probes are hybridized). The (logarithm of the) ratio between the saturation intensity and the detection limit is known as the “dynamic range”.

Suzuki et al. (BMC Genomics, 2007, 8:373) have studied the optimal probe length for detecting single nucleotide mismatches based on a custom designed array with probes of different length (14- to 25-mer) and all possible single nucleotide mismatches and describe hybridization with artificial 25-mer oligo-DNAs as targets. The authors plotted the average intensity for each probe length and observed that longer probes resulted in an increased risk of saturation of signal intensities. In a follow-up study, Ono et al. (Bioinformatics, 2008, 24: 1278-1285) introduced an extended thermodynamic model of hybridization to improve the dynamic range of measurements, using a custom microarray using perfect match probes of varying length, which were perfectly complementary to the artificial target sequences.

US20080131892 discloses methods to extend the dynamic range using a probe reagent comprising two or more probes, wherein the probes have e.g. a different probe length, and wherein the total hybridization intensity for the mixture of probe is measured.

It is of importance to obtain a dynamic range which is as high as possible in order to reliably measure different potential hybridization intensities for a sample in a single experiment, and thus to have more reliable detection methods which are less sensitive to variation in sample concentration and hybridization conditions.

### SUMMARY OF THE INVENTION

It is an aim of the present invention to provide detection methods based on nucleotide hybridization which provide an increased dynamic range. The present inventors have found that this can be achieved through the use of two or more probe sets, which differ in the length of the hybridization sequence of the probes. More particularly, the methods make use of probe sets comprising probes with a hybridization sequence of a fixed length with varying complementarity to the target sequence which hybridization sequence in the different probe sets is extended by one or more nucleotides. More particularly, detection of a target polynucleotide comprising a target sequence is provided using two or more probe sets designed based on said target polynucleotide sequence, each probe set comprising a plurality of different probes each comprising a core sequence of uniform length corresponding to (part of) said target sequence, wherein said core sequences of said probes are characterized by a varying complementarity to said target sequence and wherein said second and optionally further probe sets set is characterized in that each probe within a probe set is identically elongated at one or both ends with a fixed number of one or more nucleotides which are complementary to the corresponding nucleotides of said target polynucleotide (whereby said fixed number is different between each of said second and further probe sets). Typically the methods will involve contacting the sample believed to comprise the target sequence with the two or more probe sets and detecting the hybridization intensities for the different probe sets.

In particular, provided herein is a method for determining the presence of a target polynucleotide comprising a target sequence in a sample solution, said method comprising: (i) contacting said sample solution with a first probe set and a second probe set and an optional one or more further probe sets, wherein the first probe set comprises a first plurality of different probes each comprising a core sequence of uniform length corresponding to said target sequence, wherein said core sequences are characterized by a varying complementarity to said target sequence; and the second probe set comprises a second plurality of different probes each comprising a core sequence identical to a core sequence of a corresponding probe of said first probe set, and each identically elongated at one or both ends with a fixed number of one or more nucleotides which are complementary to the corresponding nucleotides of said target polynucleotide; the method further comprising obtaining hybridization intensities of said sample solution for said first, second and optional one or more further probe sets and (ii) determining the presence of said target polynucleotide based on the combined hybridization intensities detected for said probes of said first probe set and said second probe set, wherein the hybridization intensities for one or more of said probe sets are rescaled to compensate hybridization intensity differences between corresponding probes of said probe sets to obtain said combined hybridization intensities.

In particular embodiments of the present method, the detection step comprises (a) identifying the probes of the first probe set and the probes of the second probe set for which the hybridization intensity is within the dynamic range of the used setup; (b) rescaling hybridization intensities for each of the probes of one or more of said probe sets to compensate hybridization intensity differences between corresponding probes of said probe sets, thereby obtaining a rescaled dataset; and (c) determining the presence of said target polynucleotide based on said rescaled dataset. In particular embodiments, said first probe set is a reference probe set and the method encompasses rescaling the hybridization intensity differences between corresponding probes of said second and optional further probe sets and said first probe set.

In certain embodiments of the present method, step (ii) comprises analyzing the hybridization intensities of said probes of said first, second, and optional further probe sets as function of the hybridization intensity of the corresponding probe of one of said probe sets; and/or the hybridization free energy for the hybridization between said target polynucleotide and the corresponding probe of one of said probe sets.

In certain embodiments, the present method further comprises identifying for each of said probe sets the probes for which the logarithm of the hybridization intensity follows a linear regime in function of said hybridization free energy and/or the logarithm of the hybridization intensity of the corresponding probe of one of said probe sets; and combining the data of the probes within each of said linear regimes.

In particular embodiments of the present method, the hybridization intensities for one or more of said probe sets are rescaled by multiplying each of the individual hybridization intensities of the probes within a single probe set by the same rescaling factor.

In certain embodiments of the present method, step (i) further comprises contacting said sample solution with at least a third probe set comprising a plurality of different probes each comprising a core sequence identical to a core sequence of a corresponding probe of said first probe set and each identically elongated at one or both ends with a fixed number of one or more nucleotides which are complementary to the corresponding nucleotides of said target polynucleotide, wherein at least one of said fixed number of nucleotides is different from said fixed number of nucleotides in said second probe set; and step (ii) comprises determining the presence of said target polynucleotide based on the hybridization intensities detected for said probes of said first probe set, said second probe set, and said third probe set.

In certain embodiments, the present method is a method of detecting the presence of a mutant polynucleotide in a sample solution, said mutant polynucleotide differing from a target polynucleotide comprising a target sequence in one or more nucleotides of said target sequence; wherein said method comprises: contacting said sample solution with probes of said first and second probe set, and optional with a third or further probe set, and obtaining first hybridization intensities for each of said probes; optionally, contacting a reference solution comprising said target polynucleotide and essentially free of said mutant polynucleotide, with said probes of said first and second probe sets, and optional of said third or further probe sets and obtaining hybridization intensities for each of said probes; and comparing said hybridization intensities of said probes of said first, second, and optional third or further probe set obtained for said sample to the hybridization free energy for the hybridization between said target polynucleotide and the corresponding probe of one of said probe sets; or to the hybridization intensities of said probes of said first, second, and optional third or further probe set obtained for said reference sample.

In particular embodiments of the present method, said core sequence of the probes of said first probe set each comprise up to two mismatches against said target sequence.

In certain embodiments of the present method, said first, second, and optional further probe sets each comprise a perfect match probe for said target sequence.

In particular embodiments of the present method, each probe of said second probe set comprises a core sequence which is identical to the core sequence of a corresponding probe of said first probe set, and each identically elongated at both ends with a fixed number of one or more nucleotides which are complementary to the corresponding nucleotides of said target polynucleotide.

In certain embodiments of the present method, each of said first, second, and third or further probe sets comprises at least 100 probes.

In particular embodiments of the present method, the core sequence of the probes of the probe sets has a fixed length between 15 and 25 nucleotides.

In certain embodiments of the present method, each of said probes of said first, second, and third or further probe set are provided on separate spots of a microarray. More particularly, the micro-array comprises only one probe type per spot.

Further provided herein is a microarray comprising a plurality of microarray spots each of them comprising a probe, said probes forming a first probe set, a second probe set and optionally a third or further probe set, wherein: said first probe set comprises a plurality of different probes each comprising a core sequence of uniform length corresponding to a target sequence within a target polynucleotide, wherein said core sequences are characterized by a varying complementarity to said target sequence; said second probe set comprises a plurality of different probes each comprising a core sequence identical to a core sequence of a corresponding probe of said first probe set, and each identically elongated with a fixed number of one or more nucleotides which are complementary to the corresponding nucleotides of said target polynucleotide; and said optional third probe set comprises a plurality of different probes each comprising a core sequence identical to a core sequence of a corresponding probe of said first probe set, and each identically elongated with a fixed number of one or more nucleotides which are complementary to the corresponding nucleotides of said target polynucleotide; wherein the fixed number (and/or position) of one or more nucleotides of the third probe set is different from the fixed number of one or more nucleotides in the second probe set.

Further provided herein is a computer program product for performing, when executed on a computing device, a method for analyzing hybridization and/or a method for determining the presence of a target polynucleotide comprising a target sequence in a sample solution as described herein.

By providing an increased dynamic range, the present methods can increase the dynamic range of hybridization analysis experiments and can therefore allow for analyzing samples under less stringent conditions compared to conventional methods for analyzing hybridization. Indeed, for any hybridization analysis experiment, a given set of experimental parameters like temperature, sample concentration and buffer constitution, the affinity window of probe-target interaction is always limited and can impose strong boundary conditions on the experiment design, especially when high parallelization is aimed for. Vice versa, for a given design, changes in experimental conditions can easily throw measurements out of detection range. Accordingly, by increasing the detection range (dynamic range), the experiments can be conducted using wider parameter ranges. For example, the present methods may be used for analyzing samples having wide concentration range of target polynucleotides.

The present methods may further allow for using a wide range of hybridization sequence lengths that can be used for hybridization experiments for analyzing a specific target polynucleotide. The above and other characteristics, features and advantages of the concepts described herein will become apparent from the following detailed description, which illustrates, by way of example, the principles of the invention.

### BRIEF DESCRIPTION OF THE DRAWINGS

The following description of the figures of specific embodiments of the methods and instruments described herein is merely exemplary in nature and is not intended to limit the present teachings, their application or uses. Throughout the drawings, corresponding reference numerals indicate like or corresponding parts and features.

FIG. 1 Illustration of the probe design used for the hybridization experiments. The probes are bound to a solid surface (6) and have a variable central region or core sequence (seqα) which differs from probe to probe. Each probe has three length representations L21 (1), L23 (2), and L25 (3). These representations only differ from each other by a number of boundary nucleotides, which are identical for all probes. The probes can hybridize to the target polynucleotide (4) which may be provided with a label (5).

FIG. 2 Plot of the theoretical Langmuir isotherm following equation 1 for all three probe sets, where intensity signals from sequences with affinity ΔG, ΔG₋, and ΔG₊ are denoted by l, l₋, and l₊, respectively.

FIG. 3 Plot of the theoretical Langmuir isotherm of FIG. 2, after shifting of the intensity signals l₋ and l₊ towards I through the distance Δ₋ and Δ₊. The linear regime has widened, demonstrating that the combination of the intensity signals from different lengths of probes for a particular sequence allows for increasing the dynamic range of the measurement.

FIG. 4 Plot of l₋, l, and l₊ against l, providing a visualization of the vertical distance among the linear data between l₋ to l, and l₊ to l. These vertical distances provide estimates of α₋ and Δ₊.

FIG. 5 Plot of l₋, l, and l₊ against l, wherein the intensities l₋ and l₊ are shifted towards l over the distances Δ₋ and Δ₊, respectively. This provides a visualization of the estimated position of the kinks in the curves (points a, b, c, and d).

FIG. 6 Plot of data from a microarray hybridization experiment to test our method. Intensity signals l₋, l, and l₊ are plotted against l. The maximum intensity is below 10⁵ while the minimum intensity is only slightly higher than 10°. From this Figure, one can estimate the distances Δ₋ and Δ₊.

FIG. 7 Plot of the same data as in FIG. 6, wherein the signals l₋ and l₊ are shifted towards l through the distanced Δ₋ and Δ₊ respectively.

FIG. 8 Plot of all intensities against estimated ΔΔG.

FIG. 9 Plot identical to the plot of FIG. 8, after removal of unnecessary data. The dynamic range marked by the linear regime has increased.

FIG. 10 A, B, C: Plot of intensity data for a solution comprising wild type and mutant (Y axis) versus intensity data for corresponding probes for a solution comprising wild type only (X axis) using probes having a length of 21, 23, and 25 nucleotides, respectively.

FIG. 11 Plot of intensity data of FIG. 10, wherein the intensity data of the probes of different lengths are combined according to a particular embodiment of the present method.

FIG. 12 Schematic representation of a particular embodiment of the methods of the present invention. FIG. 12.A represents the target polynucleotide showing the target sequence (white box) and two nucleotides (◯) of the target polynucleotide flanking the target sequence. FIG. 12.B shows the hybridization sequences of 3 sets of probes (1), (2) and (3), wherein each set comprises a plurality of probe sequences; probe set (1) comprise probes having a hybridization sequence (gray box) of the same length as the target sequence and with a varying complementarity to the target sequence, i.e. at least one probe hybridization sequence is a perfect match to the target sequence, while other probe hybridization sequences in the probe set have one or two mismatches (represented by the arrow). Probe sets (2) and (3) consist each of a plurality of probes wherein each individual probe has a hybridization sequence (gray box) consisting of the hybridization sequence (gray box) of a probe of probe set (1) elongated with the identical one [as in probe set (2)] or two [as in probe set (3)] nucleotides () which are complementary to the nucleotides (◯) of the target polynucleotide flanking the target sequence. FIG. 12.C shows the plot of the linear regime of the hybridization intensities for hybridization of one or more polynucleotides in the sample solution with the probes of the different probe sets vs the ΔΔG or vs the hybridization intensity for hybridization with probe set (2). FIG. 12.D shows that after shifting the plots of (1) and (3) [i.e. rescaling of (1) and (3)] and combining it with plot (2), a combined data set is obtained with increased dynamic range.

In the figures, the following numbering is used:

1, 2, 3—probe; 4—target; 5—label; 6—surface.

### DETAILED DESCRIPTION OF THE INVENTION

While potentially serving as a guide for understanding, any reference signs used herein and in the claims shall not be construed as limiting the scope thereof.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” when referring to recited components, elements or method steps also include embodiments which “consist of” said recited components, elements or method steps.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments described herein are capable of operation in other sequences than described or illustrated herein.

The values as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to ensure one or more of the technical effects envisaged herein. It is to be understood that each value as used herein is itself also specifically, and preferably, disclosed. Typically, the term “about” should be read in this context.

The recitation of numerical ranges by endpoints includes all numbers and fractions subsumed within the respective ranges, as well as the recited endpoints.

All documents cited in the present specification are hereby incorporated by reference in their entirety.

Unless otherwise defined, all terms used in disclosing the concepts described herein, including technical and scientific terms, have the meaning as commonly understood by one of ordinary skill in the art. By means of further guidance, definitions for the terms used in the description are included to better appreciate the teaching of the present disclosure. The terms or definitions used herein are provided solely to aid in the understanding of the teachings provided herein.

The term “polynucleotide” as used herein may include oligonucleotides and refers to polymer composed of nucleotide monomers, typically having a length of at least 10 nucleotides. Typically, the polynucleotides such as the target polynucleotides and probes referred to herein are single-stranded polynucleotides. As used herein, the term “polynucleotide” may include deoxyribonucleic acid (DNA), ribonucleic acid (RNA) or peptide nucleic acid (PNA).

The term “equilibrium” as used herein refers to thermodynamic equilibrium and indicates a situation wherein a steady state is obtained such that the number of conventional target-probe bindings does not substantially change over time. The term “non-equilibrium” or “non-equilibrium effects” refers to occurrence of a target-probe binding state that may change over time.

The term “free energy” as used herein refers the Gibbs free energy (ΔG) or chemical potential. Where in embodiments analysis is performed as function of hybridization free energy, this includes analysis as function of ΔΔG, being the free energy difference between a perfect matching hybridization and a hybridization where the probe sequences have one or more internal mismatches.

The term “hybridization” as used refers to nucleic acid hybridization. This refers to the process of establishing a non-covalent sequence-specific interaction between two or more complementary strands of nucleic acids into a single hybrid. The strands of nucleic acids that may bind to their complement can for example be oligonucleotides, DNA, RNA or PNA. Nucleotides form the basic components of the strands of nucleic acids. Hybridization comprises binding of two perfectly complementary strands (in the Watson-Crick base-pairing senses), but also binding of non-perfect complementary strands. With a non-perfect complementary strand reference may be made to strands having a small number of non-complementary elements such as one, two or more non-complementary elements, preferably one or two non-complementary elements. In principle there is no limit to the number of non-complementary elements but the more non-complementary elements, the easier these are detectable.

Reference throughout this specification to “one embodiment” or “an embodiment” means that a particular feature, structure or characteristic described in connection with the embodiment is included in at least one embodiment envisaged herein. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are also envisaged herein, and form different embodiments, as would be understood by those in the art. For example, in the appended claims, any of the features of the claimed embodiments can be used in any combination.

Provided herein are methods for analyzing hybridization. More particularly, the present methods may allow for analyzing the hybridization between a probe and a target polynucleotide and/or a mutant thereof. The target polynucleotide is not limited to specific nucleotide types and may for example comprise DNA, RNA, or PNA.

Although the present description focuses on the interaction between a strand that initially is in a sample solution, and a strand that is bound to a surface, it is noted that the present methods are not limited thereto. Accordingly, hybridization may occur between nucleic acid strands that both are in solution.

The strands in the sample solution are typically referred to in the art as “target”, whereas the strand provided to hybridize to the target (or a mutant thereof) is referred to as “probe”. Accordingly, the target polynucleotide or mutants thereof referred to herein may be considered as “targets”. The probe may for example be a strand of oligonucleotides, DNA, RNA or PNA (partially) complementary to a target which may be present in the sample solution. In particular embodiments, the target probes are bound to a surface.

The term “mutant polynucleotide” or “mutant” as used herein refers to a polynucleotide having a sequence which differs from the sequence of the target polynucleotide in one or more nucleotides. It will be understood by the skilled person that the term “mutant” is not limited to sequences which are the result of a change in the target polynucleotide in a specific organism, tissue or cell but also include naturally occurring (i.e. evolutionary) sequence variants. Typically, these differences or mutations are located within a certain subsequence of the target polynucleotide, referred to herein as the “target sequence”.

Again, it will be understood that the “target sequence” is the sequence used as the reference sequence in certain embodiments of the present invention. In particular embodiments, the mutant polynucleotide only differs from the target polynucleotide in one or more nucleotides within the target sequence. Preferably, the mutant polynucleotide differs from the target polynucleotide in a limited number of nucleotides, preferably in at most two nucleotides, such as only in one nucleotide.

The methods described herein comprise measuring the degree of hybridization between two or more probe sets and a sample solution, wherein the probe sets are characterized by probes having different hybridization sequence lengths. The hybridization sequence of a probe is also referred to herein as “probe sequence”. It is noted however, that the probe may contain other parts such as a tail sequence (see further) and/or a linker for coupling the probe to a solid surface.

By combining hybridization intensity data of the two or more probe sets, the dynamic range of the hybridization experiment can be increased significantly. More particularly, in a first aspect, the present application provides methods for analyzing hybridization between a target polynucleotide comprising a target sequence (or a mutant thereof) in a sample solution, and a probe. The present methods comprise:
(i) contacting the sample solution with corresponding probe sets comprising a first probe set, a second probe set and optionally a third or further probe set, wherein the probes of different probe sets differ from each other in the hybridization sequence length; and
(ii) obtaining and analyzing hybridization intensities for hybridization of one or more polynucleotides in the sample solution with the probes of the first probe set, the second probe set, and optionally the third or further probe set; more particularly determining the presence of the target polynucleotide based on the hybridization intensities detected for the probes of the first probe set and the second probe set.

Thus, the present methods comprise contacting the sample solution to at least a first and a second probe set. Optionally, the sample solution may be contacted a third probe set, or even further probe sets. As used herein, the term “probe sets” may refer to the first and second probe sets only; or to all probe sets of the experiments. In preferred embodiments, the sample solution is contacted simultaneously to the different probe sets. For example, the probe sets may be provided on a single microarray, more in particular each of said probes of said first, second, third or further probe set are provided on separate spots of a microarray. However, it is envisaged that in certain embodiments, the sample solution is not contacted simultaneously to the different probe sets.

The contacting of the sample solution to the probe sets is typically performed under conditions suitable for hybridization of the target polynucleotide to said probes. These conditions are typically also suitable for hybridization of mutants to the probes, given the similarity between the target polynucleotide and mutant polynucleotide. The skilled person understands that relevant parameters for optimizing hybridization include hybridization time, temperature, and hybridization sequence length. In particular embodiments, the hybridization sequence of the probes of the first probe set has a fixed length between 15 and 25 nucleotides.

The probes can hybridize to the target polynucleotide or mutants because the probes each contain a “hybridization sequence”. This is a polynucleotide sequence which is complementary to the target sequence of the target polynucleotide, optionally with the exception of one or more mismatches. The probes of a single probe set are characterized in that their hybridization sequences all have the same length, i.e. the number of nucleotides in the hybridization sequence is uniform (fixed) within each probe set. Each of the probe sets is characterized by a different hybridization sequence length of the probes. In the present methods, the hybridization sequence of the probes of the first probe set is shorter than the hybridization sequences of the other probe sets. The hybridization sequences of the probes of the first probe sets will also be referred to herein as “core sequences”. The core sequences have the same length (i.e. the same number of nucleotides) as the target sequence, and are selected such that they provide a varying complementarity to the target sequence.

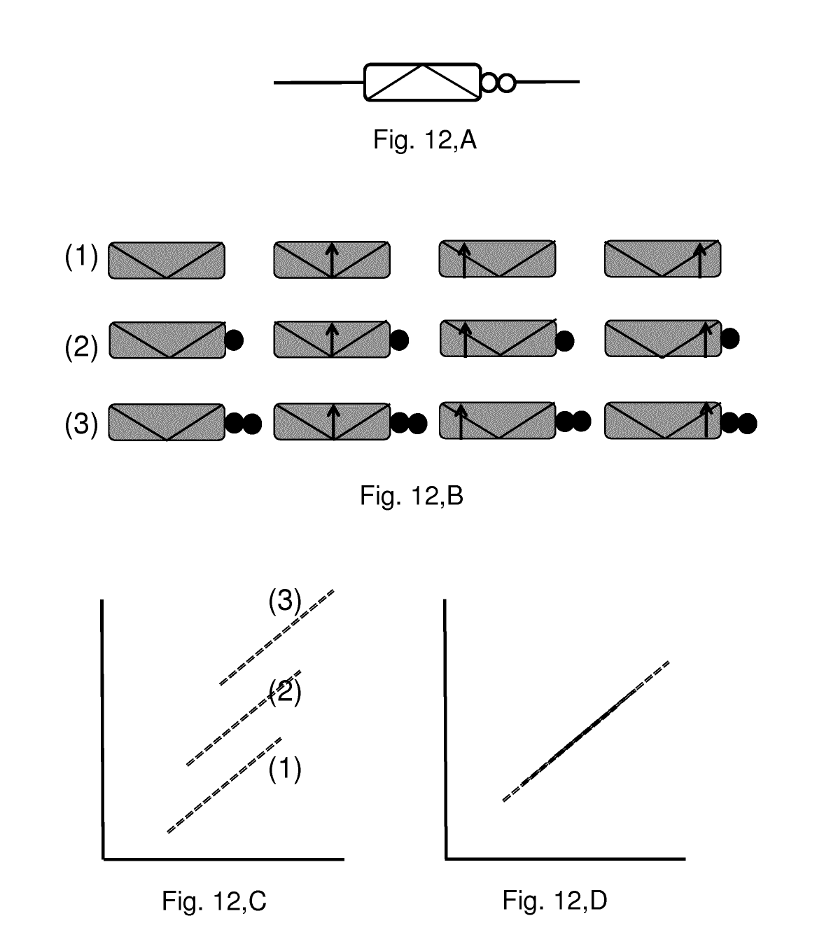

In the present methods, the hybridization sequence length of the probes of the second probe set is at least one nucleotide longer than the core sequence length. Similarly, the hybridization sequence length of the probes of the third probe set (if present) is at least one nucleotide longer than the core sequence, and preferably also at least one nucleotide longer compared to hybridization sequence length of the probes of the second probe set; and so on, as schematically represented in FIG. 12.

The probe sets are further characterized in that for each probe of the first probe set having a certain probe sequence, there is a probe in each of the other probe sets having an identical core sequence, wherein this core sequence is further elongated with one or more additional nucleotides at one or both ends of the core sequence. Such probes are referred to herein as “corresponding probes”. The additional nucleotides are further identical for all probes within a single probe set, and are complementary to the corresponding nucleotides of the target polynucleotide. Stated differently, the non-complementary or mismatch elements between the different probes sets and the target nucleotide occur only in the core sequence. This is schematically represented in FIG. 12.

In preferred embodiments, the probe sets are characterized in that for each of the hybridization sequences of probe set (n), there is a corresponding probe in probe set (n+1) (if present) containing the same hybridization sequence, except that the hybridization sequence (of probe set (n)) is further elongated with one or more additional nucleotides at one or both ends of the hybridization sequence (of probe set (n)), wherein these additional nucleotides are identical for all probes within probe set (n+1), and are complementary to the corresponding nucleotides of the target polynucleotide. It will be understood that, while the number and position of additional nucleotides is fixed within one probe set, where further probe sets are used, such as a third, fourth, fifth or further probe set, the number and/or position of the additional nucleotides should differ between the different probe sets.

In view of the above, the probe sets are characterized in that:
the first probe set comprises a (first) plurality of different probes each comprising a core sequence of uniform length corresponding to the target sequence, wherein these core sequences are characterized by a varying complementarity to the target sequence;
the second probe set comprises a (second) plurality of different probes each comprising a core sequence identical to the core sequence of a corresponding probe of the first probe set, wherein for each probe of the second probe set the core sequence is identically elongated with one or more fixed nucleotides which are complementary to the corresponding nucleotides of the target polynucleotide; and
the (optional) third probe set comprises a (third) plurality of different probes each comprising a core sequence identical to the core sequence of a corresponding probe of the first probe set, wherein for each probe of the third probe set the core sequence is identically elongated with one or more fixed nucleotides which are complementary to the corresponding nucleotides of the target polynucleotide; wherein at least one of these fixed nucleotides is different from said fixed nucleotides of the second probe set, i.e. the number and/or position (i.e. which end of the hybridization sequence) of these fixed nucleotides is different from said fixed nucleotides of the second probe set.
the optional probe set (n+1) comprises a (n+1)th plurality of different probes each comprising a core sequence identical to the core sequence of a corresponding probe of the first probe set, wherein for each probe of the probe set (n+1) the core sequence is identically elongated with one or more fixed nucleotides which are complementary to the corresponding nucleotides of the target polynucleotide; wherein at least one of these fixed nucleotides is different from said fixed nucleotides of any other probe set, including the second or third probe set, i.e. the number and/or position (i.e. which end of the hybridization sequence) of these fixed nucleotides is different from said fixed nucleotides of any other probe set.

The provision of such probe sets allows for comparing the hybridization intensities of corresponding probes of the probe sets, and can be used for increasing the dynamic range of the hybridization experiment (see further).

Thus, the core sequences of the probes of the second and (optional) third or further probe sets are identical to the core sequences of the corresponding probes of the first probe sets, wherein the core sequences of the second and (optional) third or further probe sets are elongated with one or more nucleotides, which are fixed within each probe set. This elongation may occur at one or both ends of the core sequence.

Accordingly, the term “hybridization sequence” as used herein, refers to a core sequence (for probes of the first probe set), or to a core sequence which is elongated with one or more nucleotides (for probes of probe sets other than the first probe set).

In particular embodiments, the hybridization sequence of each probe of a probe set (n+1) may be identical to the hybridization sequence of the corresponding probe of probe set (n), elongated at both ends with one or more fixed nucleotides which are complementary to the corresponding nucleotides of said target sequence.

It will further be evident to the skilled person that it is preferred that the nucleotides of the probes outside the hybridization sequences do not match with to the corresponding nucleotides of the target sequence. In this way, it can be ensured that the different affinity of corresponding probes to the target polynucleotide essentially derives only from the differences in the hybridization sequences (see further, equation 4).

As indicated above, the hybridization sequences of the probe sets are selected such that they provide a varying complementarity to the target sequence, more particularly so as to cover a range of hybridization intensities for the hybridization between the target polynucleotide and the probes. This may be obtained by providing different (single-stranded) probes having different binding affinities for the (target sequence of) the target polynucleotide. A hybridization probe may contain a hybridization sequence (intended for hybridization with the target and of which the sequence will be determined by the target) and a tail sequence, which may be used to hybridize to other sequences, for tagging of the probe, etc. Typically, the probe sets will include a plurality of mismatch (MM) probes having a core sequence or hybridization sequence having one or more, preferably up to two (i.e. one or two), non-complementary nucleotides with respect to the target sequence. Accordingly, in certain embodiments of the present methods (FIG. 12), the first, second and optional further probe set each comprise:
a perfect match (PM) probe for the target sequence; and
a variety of mismatch (MM) probes with respect to the target sequence;
   wherein said perfect match probe and said each of said plurality of probes are provided on spatially separate spots. The term “perfect match probe” as used herein refers to a probe having a hybridization sequence which is completely complementary to the target sequence of the target polynucleotide. The term “mismatch probe” as used herein refers to a probe having a hybridization sequence which is non-complementary to the target sequence because the hybridization sequence comprises one or more non-complementary nucleotides with respect to the target sequence. In preferred embodiments, the MM probes comprise at most two non-complementary nucleotides. Thus, the MM probes preferably comprise either one or two non-complementary nucleotides.

The hybridization sequence of the probes of the first probe set and the target sequence typically have the same length, i.e. contain the same number of nucleotides. Consequently, the hybridization sequence of the probes of the second and optional further probe sets will be longer than the target sequence, wherein the “additional” nucleotides of the hybridization sequence are complementary to the corresponding nucleotides of the target polynucleotide. It will be understood by the skilled person that these corresponding nucleotides are located directly next to one or both ends of the target sequence.

The present methods are of particular interest for determining the presence of a mutant polynucleotide in a sample. In these embodiments, a sequence within the target polynucleotide where the mutation is expected to occur is used as the target sequence. In certain embodiments, one of the probes of the first probe set may be designed to be complementary to the target sequence in the mutant polynucleotide.

The sample solution typically comprises the target polynucleotide and/or one or more mutants thereof, and may be prepared using standard methods known in the art.

This may include extracting DNA or other polynucleotides from a sample of interest, followed by amplification of certain fragments within the extracted DNA. Typically, amplification is performed using PCR (polymerase chain reaction). However, this results in double stranded DNA, whereas single-stranded DNA is preferred for the present methods. Indeed, hybridization of double-stranded DNA with nucleic acid probes is hampered by competition between the complementary non-target strand and the probe. Such competition can be avoided by degradation of the complementary strands, for example using lambda exonuclease. Lambda exonuclease is a processive enzyme that acts in the 5′ to 3′ direction, catalyzing the removal of 5′ mononucleotides from duplex DNA. The preferred substrate is 5′-phosphorylated double stranded DNA. Accordingly, in certain embodiments, the preparation of the sample solution may comprise the steps of:
extracting DNA from a sample of interest;
amplification of a target polynucleotide and mutants thereof contained in said DNA using a primer having a phosphate modification at its 5′ end; and
digesting said primer using lambda exonuclease.

The optimal number of probes required for the present methods may depend on various parameters such as the target sequence length and the amount and type of possible mutants expected in the sample. Typically, the first, second, and third probe sets will each comprise at least 100 different probes, preferably at least 500, at least 1000, or even more different probes.

In the methods envisaged herein, the probes may be provided on a solid surface or in solution. In preferred embodiments, the probes are provided on a surface. Although the probes may be provided on any type of carrier, it is preferred that the probes are provided on a microarray. Thus, in particular embodiments of the methods described herein, the probes of the probe sets are provided on separate spots of a microarray. A microarray as a hybridization platform contains a large number of probes which are immobilized on a solid surface. The probes are provided in spatially separated spots, wherein each spot comprises one (and only one) type of probe. Typically, each spot comprises only a few picomoles of each probe. Typical microarrays comprise hundreds or even thousands of spots. A plurality of microarray platforms suitable for use in the present methods are commercially available, and include but are not limited to the platform provided by Agilent, the GeneChips platform from Affymetrix or CodeLink Bioarray platform from Amersham Biosciences. In preferred embodiments, the different probe sets are all provided on the same microarray. This facilitates comparing the hybridization intensities for various probe sets. In other embodiments, the probes may be provided in solution. In such embodiments, each probe may be provided in a separate solution.

In hybridization based methods, the hybridization intensity is a value representing the fraction of a certain probe which is hybridized. As used herein, the term “hybridization intensity” refers to the intensity of a given, individual probe as measured during the experiment. Accordingly, the hybridization intensity may include a contribution from aspecific binding (see further, equation 1).

In the methods described herein, detection of hybridization intensity may be performed using a marker associated with the formed hybrid, such as for example a fluorescence marker or a radio-active marker, or other markers known in the art.

Typically in hybridization experiments, intensity of the radiation or fluorescence provided by the markers is detected and representative for the number of hybrids formed. Thus, in certain embodiments, the hybridization intensities may be induced by emission of a label associated with a hybrid formed by binding of the target polynucleotide or mutant thereof and the probes. Suitable fluorescence markers for the present methods include, but are not limited to, Cy3 and Cy5, which are dyes of the cyanine dye family.

The markers or labels may be associated to the target or mutant polynucleotide prior to or after hybridization. In some embodiments, a fluorescent dye or other marker compound may be associated directly to the target or mutant thereof. In other embodiments, the marker compounds may be associated to the target or mutant thereof in an indirect manner, for example via a “barcode”, which is a strand having a hybridization sequence which is complementary to a tail sequence which is present on the mutant polynucleotide of interest and on the target polynucleotide, thereby allowing hybridization between the barcode and target (or mutant thereof), and therefore indirect coupling of the fluorescence marker or other marker to the target. More particularly, the strand hybridizes to a tail sequence outside the target sequence of the target polynucleotide, such that it does not significantly interfere with the hybridization between the targets and the probes.

However, detection of hybridization intensity may also be performed without using markers, as is known by the skilled person. For example, probe-target hybridization may also be detected based on mass measurements, surface plasmon resonance measurements, impedance measurements, etc. Accordingly, the present methods are not limited to the detection of hybridization intensity using markers.

In an analysis step (ii) of the present methods, the hybridization intensities for hybridization of one or more polynucleotides in the sample solution with the probes of the different probe sets are analyzed. More particularly, analyzing the hybridization intensities of corresponding probes of the different probe sets allows for increasing the dynamic range of hybridization experiments.

Specific ways to increase the dynamic range will be explained using theoretical concepts based on the thermodynamics of DNA hybridization. The skilled person will understand that this also applies, mutatis mutandis, to nucleotides other than DNA. More particularly it is detailed herein how the hybridization intensities of the different probe sets can be combined to increase the dynamic range of the assay.

In a microarray data analysis, each probe intensity (hybridization intensity) is associated to a signal from a spot. A spot is a local space on the microarray slide that contains a large number of identical sequences corresponding to a certain type of probe in the probe set. Therefore, each spot represents a single type of probe. Each of these identical sequences within a spot is supposed to be hybridized to a floating target sequence depending on the affinity between the two sequences. This affinity is sequence dependent and determines the fraction of hybridized probes in a spot.

When nucleic acid targets hybridize on immobilized surface probes, the equilibrium state can be described by the Langmuir isotherm (Hooyberghs et al., Nucleic Acids Res. 2009, 37, e53). Assuming that the fraction of hybridized probes in a microarray spot is proportional to the detected spot intensity, for a DNA double helix in thermodynamic equilibrium, the isotherm can be written as:

l=max[l₀,A·(c·e^{-ΔG/RT})/(1+c·e^{-ΔG/RT})] (1)

where l is the total detected intensity for a given spot, l₀ the lower detection limit of the readout, A the maximum intensity when a spot is saturated by target molecules (this is a device dependent factor), c the target concentration in solution, R the ideal gas constant, T the experimental temperature and ΔG is the hybridization free energy which determines the affinity of target-probe duplexes in a sequence dependent way. Since in microarray experiments the temperature is fixed, a spot intensity remains dependent on the concentration of the sample target and the free energy of the target-probe duplex. If the target concentration is too high or the duplex affinity too strong, i.e. c·e^{-ΔG/RT}>>1, the signal will hit the saturation limit thus l≈A. If the concentration is too low or the affinity is too weak, i.e. A·c·e^{-ΔG/RT}<<l₀, the signal cannot exceed the background noise thus l≈l₀. These limits

l₀<<A·c·e^{-ΔG/RT}<<A (2)

define the “dynamic range” of any hybridization sensor. Within this range a device is in its working regime and equation 1 can be approximated by:

l≈A·c·e^{-ΔG/RT} (3)

For a given concentration c of the target polynucleotide (and/or mutants thereof) in the sample solution, the only variable in equation 3 is the free energy ΔG which differs from probe to probe. It is clear that the highest affinity (and consequently intensity l) will obtained by the PM probe which is perfectly matching the target (if present). Therefore we can shift the origin of the free energy scale to the PM probe, i.e. we define:

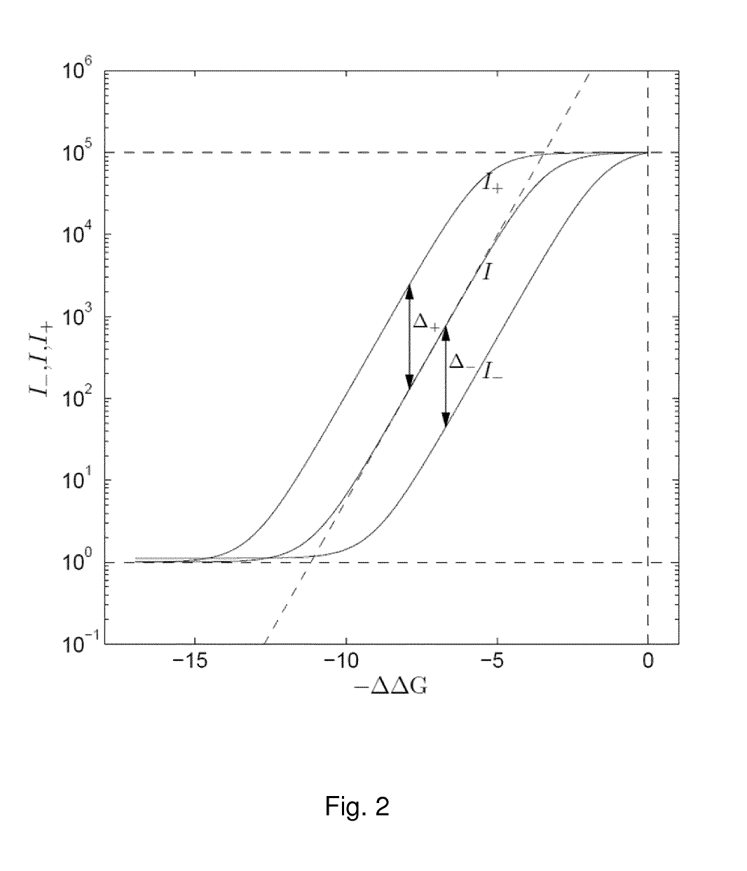

−ΔΔG(α)=−ΔG(α)+ΔG_{PM} (4)

where the arguments a and PM indicate a certain probe sequence seqα and the PM probe, respectively. With this notation the Langmuir isotherm (1) takes on the form of the central line in FIG. 2, which is a theoretical figure produced using realistic values for the variables. It is noted that the PM probe need not actually be present in the probe sets to perform this shift.

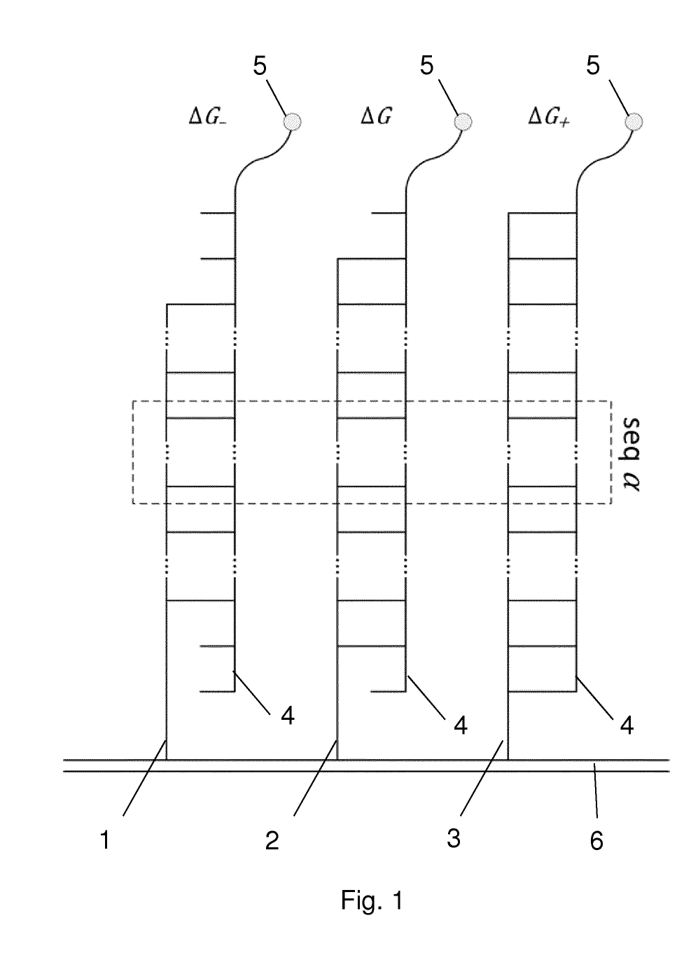

As indicated above, the present methods involve the use of two or more probe sets differing from each other in the hybridization sequence length. FIG. 1 shows three length representations of a probe, more particularly corresponding probes (1, 2, 3) from three probe sets, having a hybridization sequence with a length of 21, 23, and 25 nucleotides, respectively. Each of the three probes comprises the same core sequence seqα which has a length of 21 nucleotides. Accordingly, the probes (1) having a length of 21 nucleotides is part of the first probe set as described above. The other probes (2, 3) can be considered being part of the second and third probe sets.

In what follows, the middle length will be taken as the reference length, i.e. equation 4 is for probes having a length of 23 nucleotides (see Table 3). However, it will be understood by the skilled person that any of the lengths may be taken as reference.

The free energy of probes with different lengths will be higher or lower compared to the reference length. However, as explained above, the free energy difference between corresponding probes of different probe sets derives from fixed boundary nucleotides and is consequently independent on the central probe sequence or core sequence (seqα). Hence we can complement equation 4 by

−ΔΔG_(α)=[−ΔG(α)−δ₋]+ΔG_{PM}

−ΔΔG₊(α)=[−ΔG(α)+δ₊]+ΔG_{PM} (5)

where the plus (+) subscript is used for the longer probes (L25), the minus (−) subscript for the shorter (L21) probes, and the δ's are positive constants representing the (fixed) free energy shift relative to the reference. The impact on measurement intensities is clear from FIG. 2, which shows that in the linear regime of the Langmuir isotherm a constant shift of free energy corresponds to a constant factor in the intensities. Indeed, from the linear regime equation 3 we get:

Δ₋=e^{-δ}^{_{−}}^{/RT}

Δ₊=e^{δ}^{₊}^{/RT} (6)

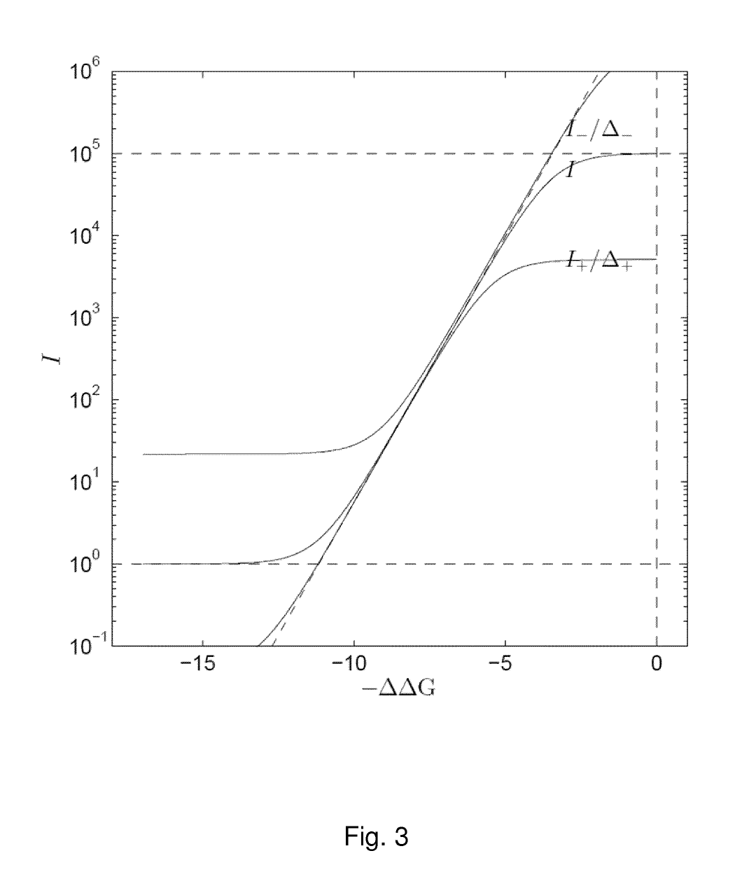

The graphs of FIG. 2 can now be replotted using l, l₋/Δ₋, and l₊/Δ₊, resulting in FIG. 3. Basically, the replot results in a shift of l₋ (intensity measured for the shortest probes L21) and l₊ (intensity measured for the longest probes L25) towards l (intensity measured for the reference probes L23), which allows for a combined use of the data of the three probe sets.

Indeed, FIG. 3 shows that the linear regime of the plot is expanded when using signal l₋/Δ₋ in the high intensity region and l₊/Δ₊ in the low intensity region. This widening of the linear regime effectively results in an increase of the dynamic range and quantitatively corresponds to Δ₊/Δ₋. For the experimental data shown in FIG. 3, this results in an increase of the dynamic range l with a factor 25.

Thus, FIG. 3 demonstrates that the dynamic range for DNA hybridization measurements can be increased by combining the data of probes having different lengths according to the present methods. It is noted that whereas FIG. 3 shows a plot of the intensities in function of −ΔΔG, real-life experiments may involve the detection only of l whereas ΔG or ΔΔG are not directly provided by the experiments. Although it is possible to use estimated values for ΔG or ΔΔG are, these values may contain statistical errors, such that the introduction of additional errors through the use of estimated values for ΔG or ΔΔG may not be desirable.

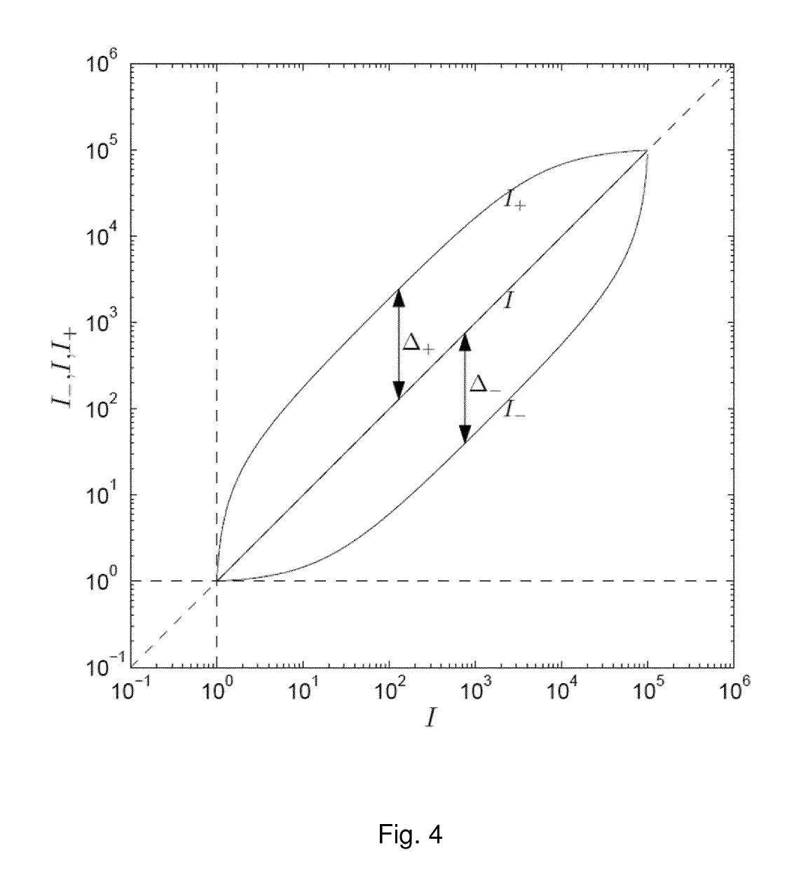

In what follows, it will be shown that it is possible to increase the dynamic range while relying only the measured intensity signals. FIG. 4 shows a plot of l₋, l, and l₊ against l. By using equation 6 one can estimate Δ₋ and Δ₊ from the intensities data as:

Δ₋=l₋/lₗ

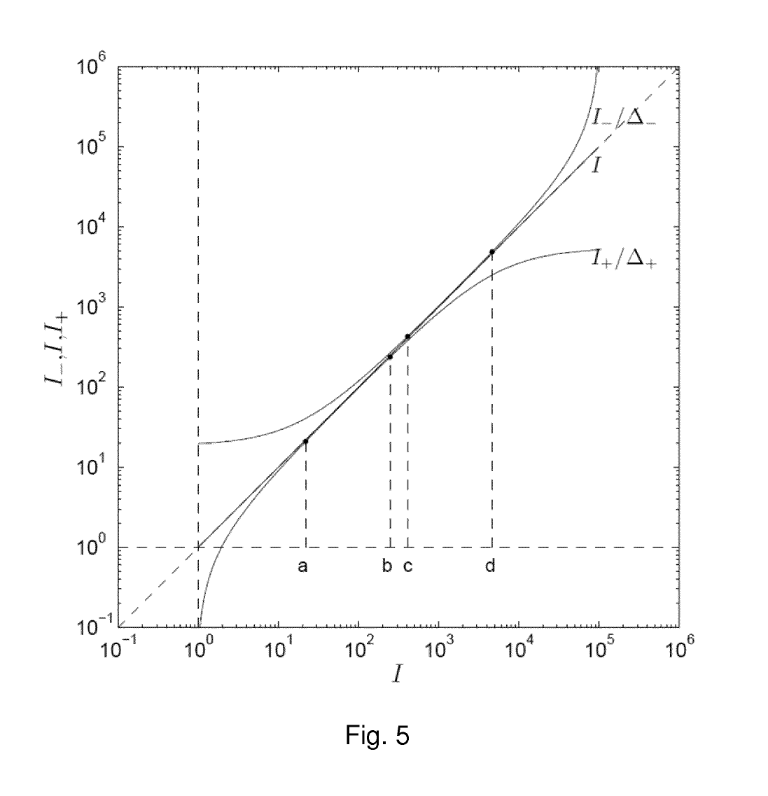

Δ₊=lₗ₊/lₗ (7)

wherein the subscript “l” refers to the linear regime of the plot. Thus, in practice the distances Δ₋ and Δ₊ can be determined by identifying subsets of data showing a linear behavior (via statistics or other methods), and then estimating the distances Δ₋ and Δ₊ for these subsets. By shifting l₋ and l₊ towards l over the distances Δ₋ and Δ₊ respectively, a shifted plot can be obtained as shown in FIG. 5.

Points for sectioning data can be estimated by finding the kinks in the intensity curves. For example four points (a, b, c, d) can be defined as shown in FIG. 5. Point a is where l₊/Δ₊ started to strongly deviates due to l is reaching the background l0. The kink in point b is due to l₋/Δ₋ quickly reaching the background. Point c is where l₊/Δ₊ starts to saturate. Point d is where l starts to saturate.

The “low intensity regime” of the experiment can be defined as any intensity region below point a and the “high intensity region” can be defined as all intensities above point d. Therefore, in the region <a we use l₊/Δ₊ signals, in the region >d we use l₋/Δ₋ signals, and l signals are used in the region between a and d.

Thus, in view of the above, certain embodiments of the methods described herein may comprise analyzing the hybridization intensities of the probes of the different probe sets as function of:
the hybridization intensity of the corresponding probe of a reference probe set, which is one of the different probe sets; and/or
the hybridization free energy for the hybridization between said target polynucleotide and the corresponding probe of one of said probe sets.

From the above, it follows that especially the logarithm of the hybridization intensities is relevant for determining Δ₋ and Δ₊. Accordingly, the present methods may involve analyzing the logarithm of the hybridization intensities of the probe sets as a function of the logarithm of the hybridization intensities of the corresponding probes of the reference probe set, which is one of the different probe sets used in the analysis. It will be evident to the skilled person that similar results can be obtained by using the actual hybridization intensities for the plot while using logarithmic scales for the axes, for example as shown in FIG. 4.

In certain embodiments, the analysis of the hybridization intensities may further comprise:
identifying for each of the probe sets the probes for which the logarithm of the hybridization intensity follows a linear regime in function of the hybridization free energy and/or the logarithm of the hybridization intensity of the corresponding probe of one of said probe sets; and
combining the results of the probes within each of said linear regimes.

More particularly, combining the results of the probes may involve shifting or rescaling the intensities of each of the probes of the second (and optional further) probe sets towards the intensities of the corresponding reference probe, as described above. In particular embodiments, the rescaling involves the step of determining the distances Δ₋ and Δ₊ (corresponding to the shift of free energy) for the probe sets compared to the reference probe set. More particularly the Δ₋ and Δ₊ can be determined by identifying subsets of data showing a linear behavior (via statistics or other methods), and then estimating the distances Δ₋ and Δ₊ for these subsets. By shifting the hybridization intensities detected for the second (and optional further) probe set(s) l₋ and/or l₊ towards l over the distances Δ₋ and Δ₊ respectively, a shifted plot can be obtained.

Thus in particular embodiments, the step of combining the data of the probes involves offsetting or rescaling the hybridization intensities for one or more of said probe sets to compensate hybridization intensity differences between corresponding probes of said probe sets. Advantageously, combining the data of the probes results in a composite data set comprising the hybridization intensities of the different probes wherein the hybridization intensities for one or more of said probe sets to is rescaled compensate hybridization intensity differences between corresponding probes of said probe sets.

It is noted that if needed, values for ΔG or ΔΔG can be determined in various ways. For example, the values may be determined experimentally or calculated. More particularly, a good estimate of these values may be obtained via the nearest-neighbor model as known by the skilled person (see Hadiwikarta W W et al., Nucleic Acids Res. 2012, 40, e138; and references cited therein).

In certain embodiments, the hybridization intensities of certain probes or spots may be excluded from the analysis. For example, probes or spots may be excluded from further analysis because the corresponding hybridization intensity is either too low (not significantly above the background signal) or too high (above the saturation level).

As a further example, certain probes or spots may be excluded from further analysis because the hybridization for these spots has not reached equilibrium. However, it is preferred that the hybridization experiments are performed under such conditions that hybridization has reached equilibrium, e.g. by selecting suitable probe lengths, temperatures, and hybridization time. A method for determining for which probes or spots hybridization has reached equilibrium is described in international patent application WO 2011/035801, which is hereby incorporated by reference in its entirety.

As indicated above, the present methods are of particular interest for determining the presence of a mutant polynucleotide in a sample. In particular embodiments, a sequence within the target polynucleotide where the mutation is expected to occur is used as the target sequence. In certain embodiments, one of the probes of the first probe set may be designed to be complementary to the target sequence in the mutant polynucleotide.

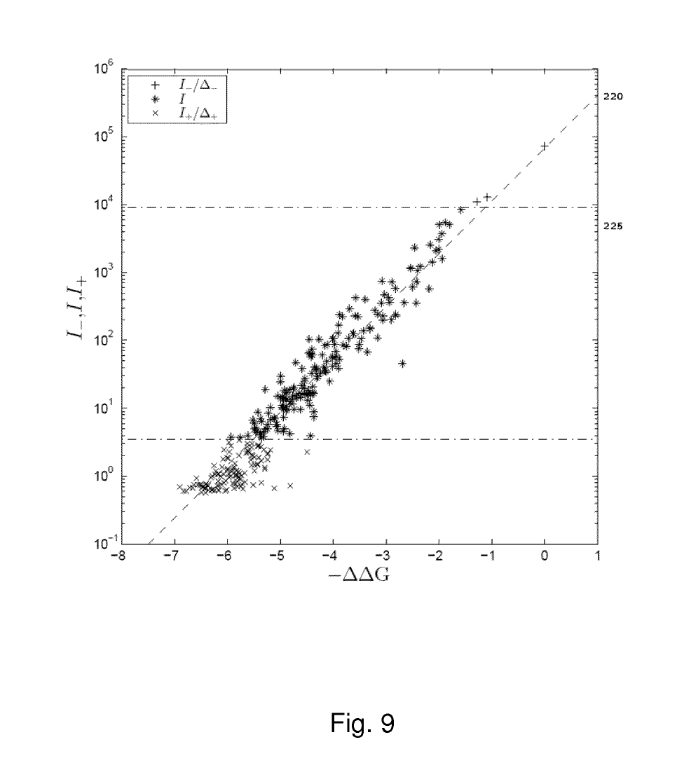

The methods may comprise comparing the hybridization intensities of the probes the different probe sets obtained for the sample solution to the hybridization free energy for the hybridization between said target polynucleotide and the corresponding probe of one of the probe sets; as described in WO2011/035801. More particularly, one of the probe sets may be considered as reference probe set. The combined hybridization intensities of the different probe sets (e.g. l, l₋/Δ₋, and l₊/Δ₊; see further) or the logarithm thereof for the probes of the different probe sets obtained for the sample may then be compared to the hybridization free energy ΔΔG of the corresponding probe of the reference probe set, as shown in FIG. 9. If the sample contains a combination of the target polynucleotide and a mutant, the plot will typically show different regimes which indicate which mutant is present in the sample.

Additionally or alternatively, the present methods may comprise a further step of contacting a reference solution comprising the target polynucleotide and essentially free of said mutant polynucleotide, with the probes of the different probe sets and obtaining hybridization intensities for each of the probes. In particular embodiments, the reference solution is essentially free of any mutant of said target polynucleotide. The hybridization intensities obtained for the sample may then be compared to the hybridization intensities obtained for the reference sample. Again, a sample containing a combination of the target polynucleotide and a mutant will typically show different regimes which indicate which mutant or mutants are present in the sample. More particularly, the (logarithm of the) combined hybridization energies of the different probe sets (e.g. l, l₋/Δ₋, and l₊/Δ₊; see further) for the sample solution may be compared with the (logarithm of the) combined hybridization energies of the different probe sets for the reference solution.

In certain embodiments, determining the presence of a mutant polynucleotide is ensured by determining whether two or more parallel linear relationships can be distinguished between parts of said logarithm of the (combined) hybridization intensities obtained for the sample solution as a function of the logarithm of the (combined) hybridization intensities obtained for the reference solution. Typically the presence of more than one linear relationship is indicative of the presence of a mutant polynucleotide of the target polynucleotide. In further particular embodiments, the methods comprise determining which of a plurality of candidate mutant polynucleotides is present in the sample solution.

Further provided herein is a microarray suitable for use in the methods described herein. More particularly, the microarray comprises a plurality of microarray spots each of them comprising a probe, said probes forming a first probe set, a second probe set and optionally a third or further probe set, wherein:
said first probe set comprises a (first) plurality of different probes each comprising a hybridization sequence of uniform length, wherein said hybridization sequences are selected so that a range of hybridization intensities for the hybridization between said target polynucleotide and the probes is covered;
said second probe set comprises a (second) plurality of different probes each comprising a hybridization sequence identical to a hybridization sequence of a corresponding probe of said first probe set, elongated with one or more fixed nucleotides which are complementary to the corresponding nucleotides of said target polynucleotide; and
said optional third or further probe set comprises a (third or further) plurality of different probes each comprising a hybridization sequence identical to a hybridization sequence of a corresponding probe of said second probe set, elongated with one or more fixed nucleotides which are complementary to the corresponding nucleotides of said target polynucleotide;

The characteristics of the probes and probe sets as described above for the present methods, are also applicable to the microarray.

Further provided herein is a computer program product for performing, when executed on a computing device, at least a part of a method for analyzing hybridization, in particular a method for determining the presence of a mutant of a target polynucleotide, as described herein. For example, the computer programs may be configured for receiving and analyzing hybridization intensities according to the methods described herein. For example, the computer program may be configured to compare the intensity of corresponding probes of the probe sets, and to identify linear regimes for each of the sets as described herein. The software may further be configured to determine the distances between the linear regimes of the various probe sets and the reference probe set, and to combine the data of the different probe sets, e.g. by shifting the data of the probe sets towards the reference probe set.

In particular embodiments, the computer program product may be configured for (i) receiving first hybridization intensities of a sample solution comprising said target polynucleotide for a first probe set, (ii) for receiving second and (optional) third or further hybridization intensities of said sample solution for a second and (optional) third or further probe set; (iii) optionally identifying the probes in the different probes sets for which the hybridization intensity is within the dynamic range of the used setup; (iv) rescaling hybridization intensities for one or more of said probe sets, e.g. by determining the distance between linear regimes of the various probe sets and subsequent shifting the data of the various probe sets by set distance, thereby obtaining a rescaled, combined data set; and (v) and determining the presence of a mutant polynucleotide in said sample solution based thereon.

In certain embodiments, the computer programs may further be configured for designing suitable probe sets based on information of the target sequence and/or mutations thereof. In case of implementation or partly implementation as software, such software may be adapted to run on suitable computer or computer platform, based on one or more processors. The software may be adapted for use with any suitable operating system. The computing means may comprise a processing means or processor for processing data.

### Examples

The following examples are provided for the purpose of illustrating the claimed methods and applications and by no means are meant and in no way should be interpreted to limit the scope of the present invention.

### 1. Sample Comprising a Single Target Sequence

### 1.1 Experimental Setup

The inventors have performed experiments involving hybridization between oligonucleotides (also referred to herein as “oligo's), more particularly a unique single-stranded DNA (ssDNA) target polynucleotide or “target” floating in solution, and probe sequences immobilized on a custom made microarray slide. The hybridization experiments were performed using custom 8×15k Agilent microarrays according to manufacturer's protocol.

In the experiments the inventors used a single target sequence at fixed concentration c, leaving as only variable free energies ΔG which differ from probe to probe. The target polynucleotide used in the experiments is shown in Table 1. The bolded nucleotides indicate the target sequence, i.e. the region where the target polynucleotide can hybridize to the probes.

Indirect labeling was used for hybridization detection. A Cy3 labeled barcode ssDNA (Table 1) was added to the hybridization mixture together with the target polynucleotide. During a single hybridization experiment the barcode hybridizes to the target and the target hybridizes to the microarray probes. The hybridization region or hybridization sequence of the barcode (Table 1, underlined) was designed to have a melting temperature above the experimental temperature which is 65° C.

Four experiments were performed, using various concentrations of target and barcode (Table 2).

TABLE 1
The two ssDNA sequences added to the hybrid-
ization mixture in the microarray experiments.
The top sequence is the target sequence, wherein
the bolded portion represents the hybridization
sequence for hybridization to the probes. The
bottom sequence is a Cy3-labeled barcode sequence,
which is added for hybridization detection. The
underlined nucleotides form the barcode
hybridization sequence.

| | |
|---|---|
| Target | 5′-CTTGCCTACGCCACCAGCTCCAACTAAAAAAAAAAT |
| | CACACAGGAAACAGCTATGACGCCAG-3' |
| | (SEQ ID NO: 1) |
| Barcode | Cy3-5'-AAAAACTGGCGTCATAGCTGTTTCCTGTG |
| | TGA-3' (SEQ ID NO: 2) |

TABLE 2
Target and barcode concentrations used in the
experiments.

| | Target | Barcode |
|---|---|---|
| Experiment | concentration | concentration |
| number | (pM) | (μM) |
| Exp. 1 | 10 | 0.5 |
| Exp. 2 | 100 | 0.05 |
| Exp. 3 | 10 | 0.05 |
| Exp. 4 | 100 | 0.5 |

TABLE 3
Sequence of the perfectly matching (PM) probe of
the three lengths used in the experiments (21,
23, and 25 nucleotides).

| Probe | Hybridization sequence | SEQ ID NO: |
|---|---|---|
| L25 | 5′-AGTTGGAGCTGGTGGCGTAGGCAAG-3′ | 3 |
| L23 | 5′-GTTGGAGCTGGTGGCGTAGGCAA-3′ | 4 |
| L21 | 5′-TTGGAGCTGGTGGCGTAGGCA-3′ | 5 |

### 1.2 Probe Set Design

Microarrays were provided containing probe sequences (1, 2, 3) having three length representations, as shown in FIG. 1. The probes provided on the microarray comprise a variable central region of interest or core sequence (seqα) which corresponds to the hybridization sequence of the shortest probe (1), wherein each of the variable seqα's occurs in each length representation. The prolongation nucleotides in the boundary of the longer probes are not variable but are completely fixed. Therefore the difference in probe-target affinity (free energy ΔG) between length representations is independent on seqα. Each collection of different seqα probes for a given probe length, i.e. each probe set, contained a probe sequence that is perfectly matching (PM) to the hybridizing region of the target (Table 3). Based on these PM probes, more probe sequences were designed, covering all possible single or double mismatches (MM) against the target hybridizing region, with the proviso that some nucleotides distance is kept between two MM's and between any MM and the border of the hybridizing region to avoid an extra free energy penalty (Hadiwikarta W W et al., Nucleic Acids Res. 2012, 40, e138). Thus, the three probe sets (L21, L23, L25) are designed such that they are measuring the same thing, and only differ by a number of additional fixed boundary Watson-Crick nucleotide pairings.

Combined, the three length representations used in the experiments comprised 1836 probes. Each of these probes was given 8 technical replicates to fill up a 15 k microarray, and the median value of the 8 replicates was used for data analysis.

### 1.3. Rescaling of Intensity Data

A number of microarray experiments were performed as described above, using three probe sets of different length. In what follows, the results from Experiment 1 (Table 2) will be discussed in more detail.

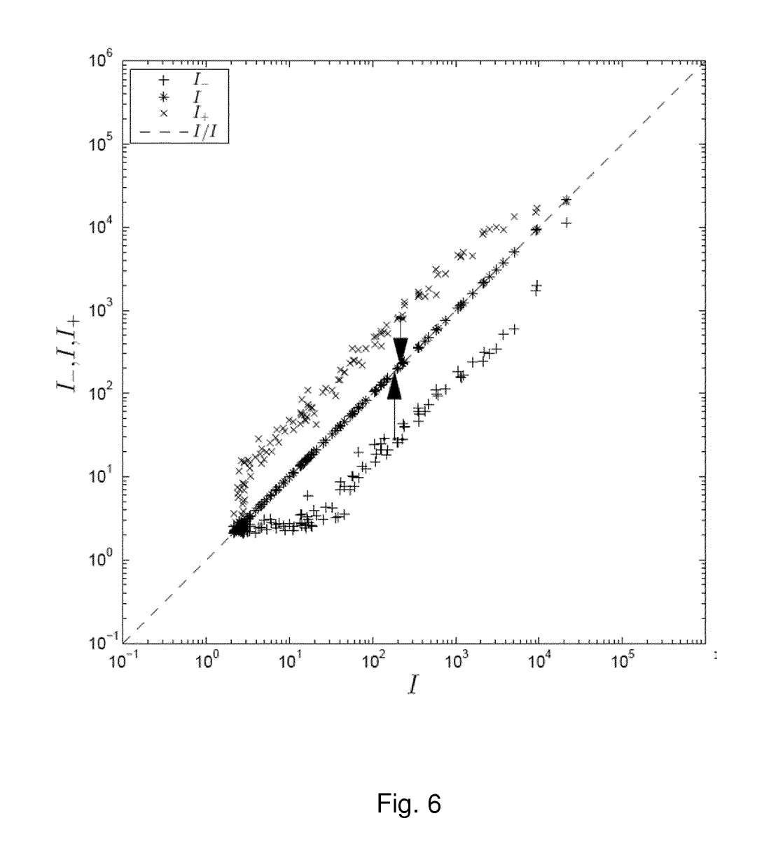

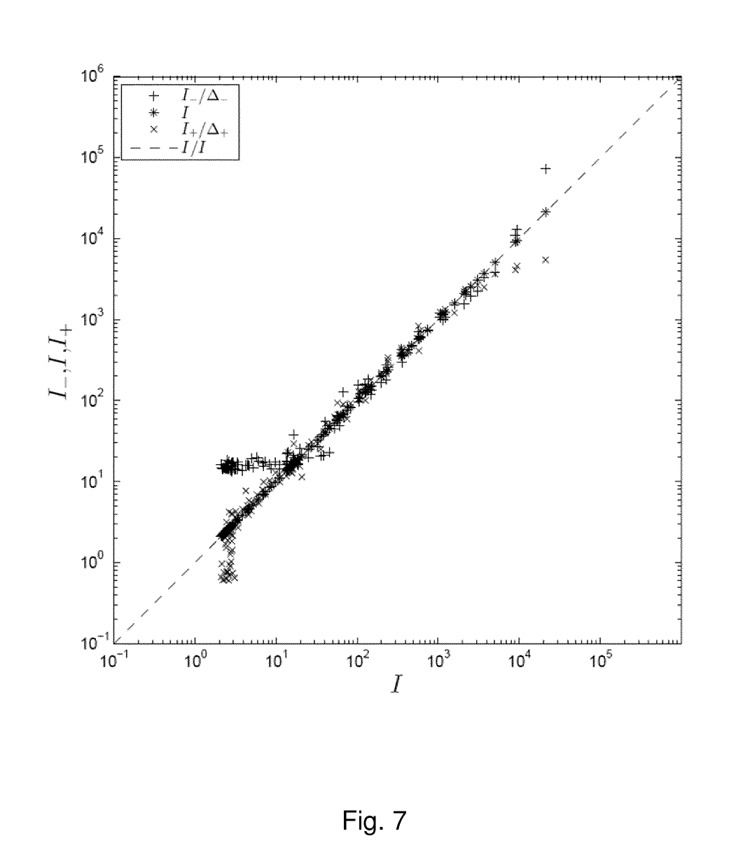

FIG. 6 shows the plots of l₋, l, and l₊ in function of l for corresponding probes (i.e. probes sharing the same seqα). Subsets of signal l₋ and l₊ having a statistically equal linear vertical distance towards the reference line l=l were identified and the average of these distances was determined. These averages correspond to distances Δ₋ and Δ₊ as defined above. The subsets of l₋ and l₊ were then shifted towards the reference line l=l over distances Δ₊ and Δ₊, respectively, thus arriving at the graph shown in FIG. 7. The distances Δ₋ and Δ₊ further allow for calculating δ₋ and δ₊ via equation 6.

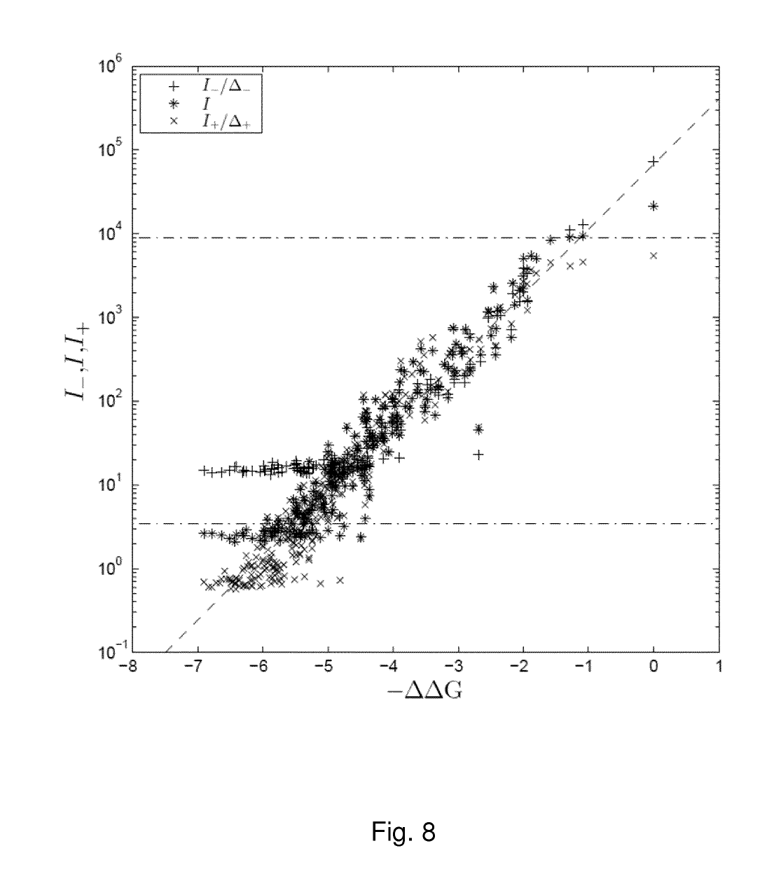

FIG. 8 shows a Langmuir isotherm, wherein the “shifted” intensities of FIG. 7 are plotted against free energy parameters as determined via the nearest-neighbor model (Hadiwikarta W W et al., Nucleic Acids Res. 2012, 40, e138).

The intensities can further be sectioned based on the low- and high intensity region as described above, using l₋/Δ₋ for the low intensity region and l₊/Δ₋ on the high intensity region, thereby obtaining data that are linear beyond the original background and saturation limit (FIG. 9). The dynamic range has increased about two orders of magnitude, which is according to the expectations for probe sets differing four nucleotides in length.

### 1.4. Mismatch and Concentration Tolerance

In hybridization-based technologies, the signal l depends on the amount of hybridized probes. As described above, from the Langmuir isotherm (equation 1), the signal l can be considered as saturated into l=A if c·e^{-ΔG/RT}>>1 and it hit the background l=l₀ if c·e^{-ΔG/RT}<<l0.

Accordingly, to have a meaningful and useful signal, the concentration c should be sufficiently high such that the signal exceeds the background, but sufficiently low as not to reach saturation. Also the free energy ΔG for the probes should be sufficiently high, which means that there should be a limited amount of mismatches between the target sequence and the probes' hybridization sequences. However, these are not the only relevant parameters for hybridization analysis. Indeed, for any hybridization analysis experiment, a given set of experimental parameters like temperature, sample concentration and buffer constitution, the affinity window of probe-target interaction is always limited and can impose strong boundary conditions on the experiment design, especially when high parallelization is aimed for. Vice versa, for a given design, changes in experimental conditions can easily throw measurements out of detection range. Accordingly, by increasing the detection range (dynamic range), the experiments can be conducted using wider parameter ranges.

In conclusion, the saturation and background levels are important factors in hybridization experiments, and limit the choice of hybridization sequences and concentration. The above results show that the methods described herein allow for increasing the dynamic range, thereby providing a significantly increased tolerance towards these two aspects. This may allow for performing hybridization experiments with samples containing lower or higher concentrations of the target polynucleotide than the concentrations used with conventional methods. The increased dynamic range can also widen the range of hybridization sequences that can be used for hybridization experiments for analyzing a specific target polynucleotide, and can increase the number of options for the design of applications that require sequence variations.

### 2. Sample Comprising Wild Type and Mutant Sequence

The method described herein can also be applied for analyzing a sample comprising both a wild type and mutant sequence. For example, the intensities obtained using a reference sample comprising only the wild type sequence may be compared to the intensities obtained with a sample comprising both wild type and mutant sequence, using identical probe sets for the two samples.

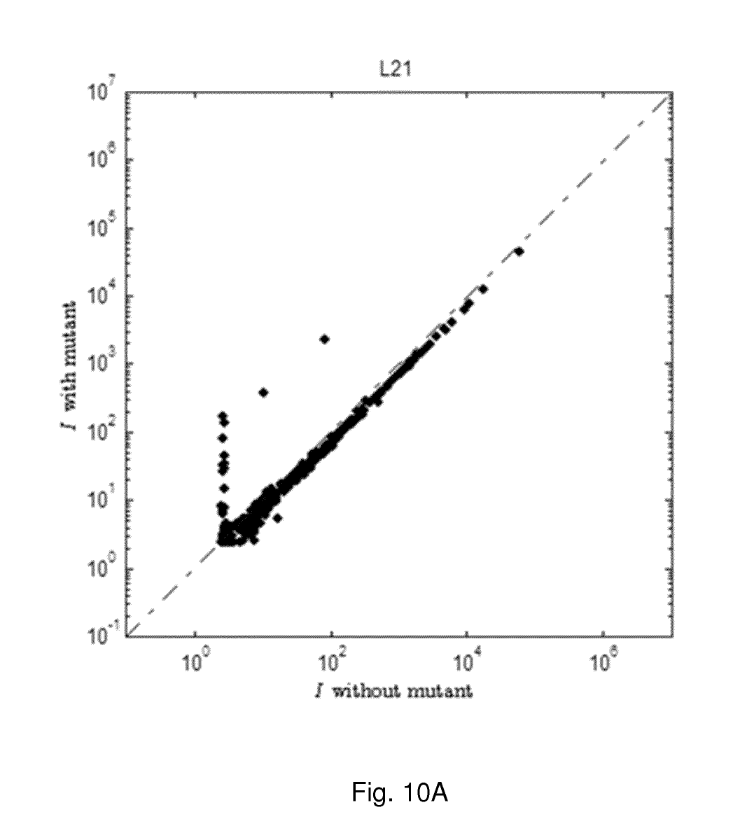

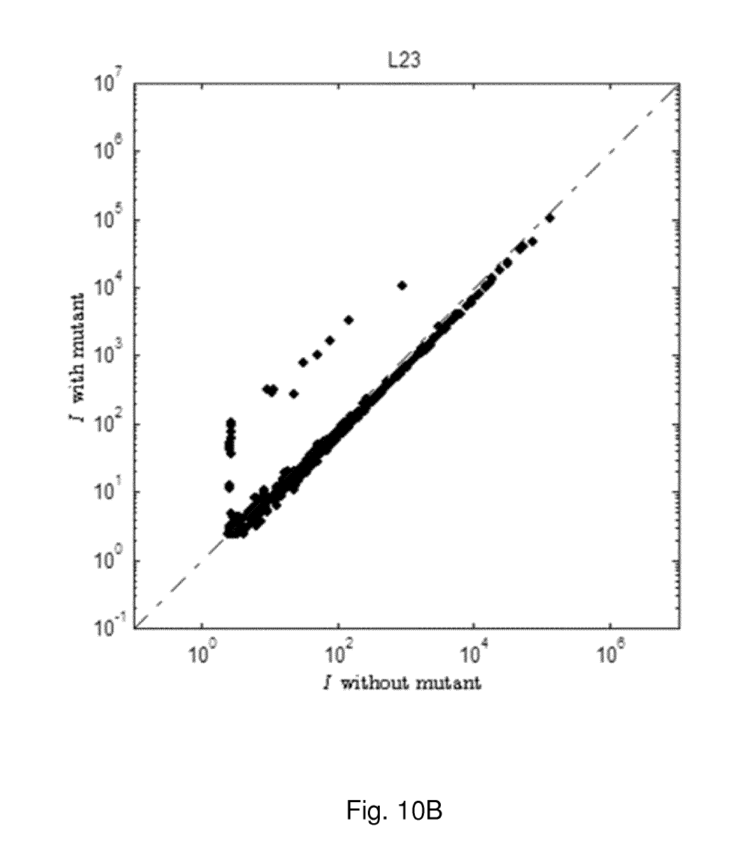

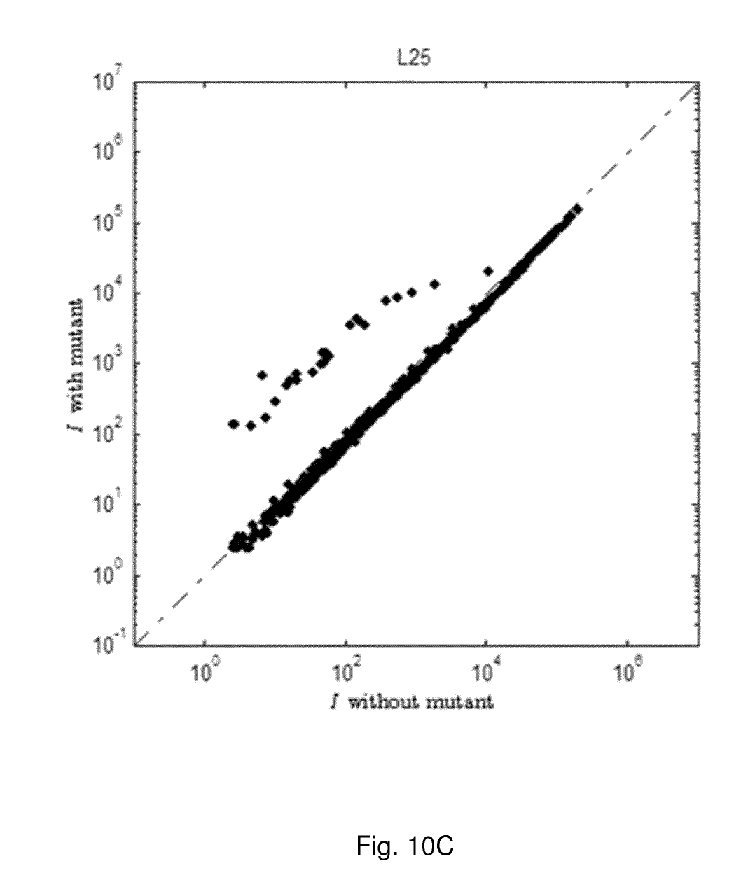

Such experiment was conducted using the target sequence of Table 1 provided above as wild type sequence, and the same probe set design comprising probe lengths of 21, 23, and 25 nucleotides. FIG. 10 shows plots of probe intensities of the sample comprising wild type sequence and mutant sequence (Y axis) versus the corresponding probe intensities of the sample comprising only wild type sequence (X axis). FIGS. 10 A, B, and C show the intensities for the probes having a probe length of 21, 23, and 25 polynucleotides, respectively.

Each plot shows a main branch and a deviating branch which is a sign of the presence of the mutant. If the system is in thermal equilibrium and within the dynamical range, the deviating branch should be a linear line parallel with the main branch. However, for none of the probe lengths a nice linear parallel deviating branch is obtained.

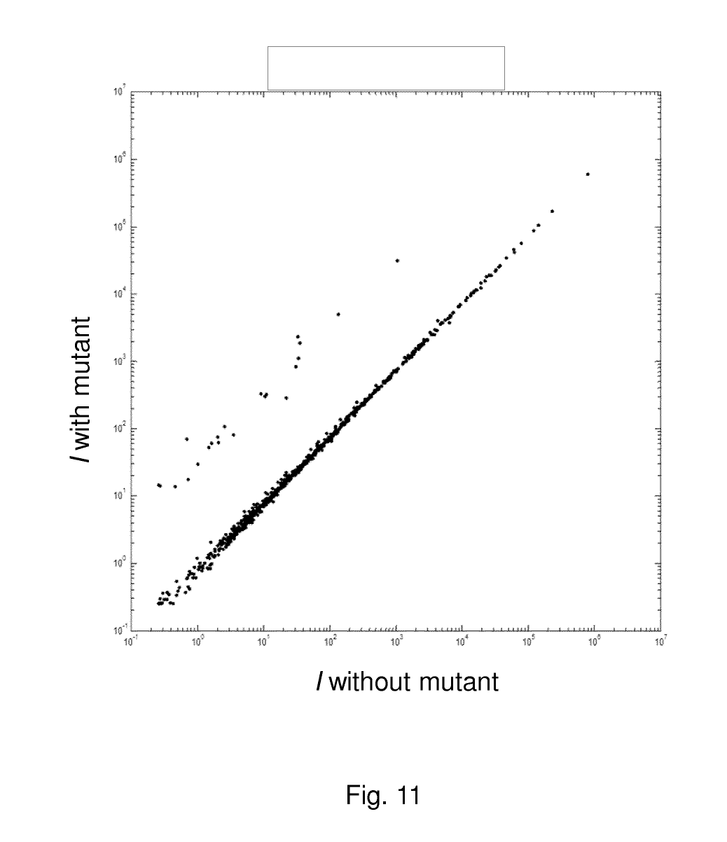

When the dynamical range extension method as described herein is applied and the information of the three probe lengths is combined, one arrives at the plot of FIG. 11, which clearly shows the extension of the dynamical range and its effect on the deviating branch. Indeed, the experimental values now follow the behaviour of equilibrium data within dynamical range. This makes data analysis much stronger since the effect of the mutation is maximized.

1-16. (canceled)

17. A method for determining the presence of a target polynucleotide comprising a target sequence in a sample solution, said method comprising:
(i) contacting said sample solution with a first probe set, a second probe set and optionally one or more further probe sets, wherein:
said first probe set comprises a first plurality of different probes each comprising a core sequence of uniform length corresponding to said target sequence length, wherein said core sequences are characterized by a varying complementarity to said target sequence;
and wherein said second probe set comprises a second plurality of different probes each comprising a core sequence identical to a core sequence of a corresponding probe of said first probe set, and each identically elongated at one or both ends with a fixed number of one or more nucleotides which are complementary to the corresponding nucleotides of said target polynucleotide; and
obtaining hybridization intensities of said sample solution for said first, second and optionally one or more further probe sets; and
(ii) determining the presence of said target polynucleotide based on the combined hybridization intensities detected for said probes of said first probe set and said second probe set, wherein the hybridization intensities for one or more of said probe sets are rescaled to compensate hybridization intensity differences between corresponding probes of said probe sets.

18. The method according to claim 17 wherein step (i) further comprises contacting said sample solution with at least one or more further probe sets comprising one or more further pluralities of different probes each comprising a core sequence identical to a core sequence of a corresponding probe of said first probe set, and each identically elongated at one or both ends with a fixed number of one or more nucleotides which are complementary to the corresponding nucleotides of said target polynucleotide, wherein at least one of said fixed number of nucleotides is different from said fixed nucleotides of any other probe set of said multiple probe sets.

19. The method according to claim 17 wherein each of said probes of said first, second or one or more further probe sets are provided on separate spots of a microarray.

20. The method according to claim 17, wherein step (ii) comprises:
(ii)(a) identifying the probes of said first probe set, the probes of said second probe set and the probes of said one or more further probe sets for which the hybridization intensity is within the dynamic range of the used setup;
(ii)(b) rescaling hybridization intensities for one or more of said probe sets to compensate hybridization intensity differences between corresponding probes of said probe sets, thereby obtaining a rescaled, combined dataset; and
(ii)(c) determining the presence of said target polynucleotide based on said rescaled, combined dataset.

21. The method according to claim 17, wherein step (ii) comprises analyzing the hybridization intensities of said probes of said first, second, and optional one or more further probe sets as function of
the hybridization intensity of the corresponding probe of one of said probe sets; and/or
the hybridization free energy for the hybridization between said target polynucleotide and the corresponding probe of one of said probe sets.

22. The method according to claim 21, further comprising:
identifying for each of said probe sets the probes for which the logarithm of the hybridization intensity follows a linear regime in function of said hybridization free energy and/or the logarithm of the hybridization intensity of the corresponding probe of one of said probe sets; and
combining the data of the probes within each of said linear regimes.

23. The method according to claim 17, wherein the hybridization intensities for one or more of said probe sets are rescaled by multiplying the individual hybridization intensities of the probes within a single probe set by the same rescaling factor.

24. The method according to claim 17, wherein
step (i) further comprises contacting said sample solution with at least a third probe set comprising a plurality of different probes each comprising a core sequence identical to a core sequence of a corresponding probe of said first probe set and each identically elongated at one or both ends with a fixed number of one or more nucleotides which are complementary to the corresponding nucleotides of said target polynucleotide, wherein at least one of said fixed number of nucleotides is different from said fixed number of nucleotides in said second probe set; and
step (ii) comprises determining the presence of said target polynucleotide based on the hybridization intensities detected for said probes of said first probe set, said second probe set, and said third probe set.

25. The method according to claim 17, which is a method of detecting the presence of a mutant polynucleotide in a sample solution, said mutant polynucleotide differing from a target polynucleotide comprising a target sequence in one or more nucleotides of said target sequence and wherein said method comprises:
(i) contacting said sample solution with probes of said first, second and optional one or more further probe set, and obtaining hybridization intensities for each of said probes;
(ii) optionally, contacting a reference solution comprising said target polynucleotide and essentially free of said mutant polynucleotide, with said probes of said first, second and optional one or more further probe sets and obtaining hybridization intensities for each of said probes; and
(iii) comparing said hybridization intensities of said probes of said first, second, and optional one or more further probe set obtained for said sample to
the hybridization free energy for the hybridization between said target polynucleotide and the corresponding probe of one of said probe sets; or
the hybridization intensities of said probes of said first, second, and optional one or more further probe set obtained for said reference sample.

26. The method according to claim 17, wherein said core sequence of the probes of said first probe set each comprise up to two mismatches against said target sequence.

27. The method according to claim 17, wherein said first, second, and optional one or more further probe sets each comprise a perfect match probe for said target sequence.

28. The method according to claim 17, wherein each probe of said second probe set comprises a core sequence which is identical to the core sequence of a corresponding probe of said first probe set, and each identically elongated at both ends with a fixed number of one or more nucleotides which are complementary to the corresponding nucleotides of said target polynucleotide.

29. The method according to claim 17, wherein each of said first, second, and optional one or more probe sets comprises at least 100 different probes.

30. The method according to claim 17, wherein said core sequence of said probes of said first probe set has a fixed length between 15 and 25 nucleotides.

31. A microarray comprising a plurality of microarray spots each of them comprising a probe, said probes forming a first probe set, a second probe set and optionally one or more further probe sets, wherein:
said first probe set comprises a first plurality of different probes each comprising a core sequence of uniform length corresponding to a target sequence within a target polynucleotide, wherein said core sequences are characterized by a varying complementarity to said target sequence;
said second probe set comprises a second plurality of different probes each comprising a core sequence identical to a core sequence of a corresponding probe of said first probe set, and each identically elongated with a fixed number of one or more nucleotides which are complementary to the corresponding nucleotides of said target polynucleotide; and
said optional one or more further probe sets comprise one or more further pluralities of different probes each comprising a core sequence identical to a core sequence of a corresponding probe of said first probe set, and each identically elongated with a fixed number of one or more nucleotides which are complementary to the corresponding nucleotides of said target polynucleotide; wherein at least one of these fixed nucleotides in said optional one or more further probe set is different from said fixed nucleotides of the second probe set and different from said fixed nucleotides of any other probe set;
wherein each of said first, second and optional one or more further probes sets comprises at least 100 different probes.

32. A computer program product for performing, when executed on a computing device, a method for determining the presence of a target polynucleotide comprising a target sequence in a sample solution according to claim 17.
